(19) United States
(12) Patent Application Publication
(10) Pub. No.: US 2010/0256060 A1
(43) Pub. Date: Oct. 7, 2010

(54) PEPTIDE PHARMACEUTICALS FOR NASAL DELIVERY

(75) Inventor: William STERN, Tenafly, NJ (US)

Correspondence Address: OSTROLENK FABER GERB & SOFFEN, 1180 AVENUE OF THE AMERICAS, NEW YORK, NY, 100368403, US

(73) Assignee: UNIGENE LABORATORIES INC., Boonton, NJ (US)

(21) Appl. No.: 12/732,081

(22) Filed: Mar. 25, 2010

Related U.S. Application Data
(60) Provisional application No. 61/166,160, filed on Apr. 2, 2009.

(51) Int. Cl. A61K 38/29; A61P 5/18

(52) U.S. Cl. 514/12

(57) ABSTRACT

Pharmaceutical products for nasal administration contain peptide active agents and are formulated with compounds that enhance bioavailability of the peptide active agents. In particular, citrates, fatty acids, sugar esters of fatty acids or acyl carnitines are used. In some embodiments, a sugar ester of a fatty acid is used in combination with either a fatty acid, or alternatively, an acyl carnitine.

### CROSS-REFERENCE TO RELATED APPLICATION(S)

This application claims priority of U.S. Provisional Application Ser. No. 61/166,160, filed Apr. 2, 2009, by William STERN, entitled PEPTIDE PHARMACEUTICALS FOR NASAL DELIVERY, the disclosure of which is incorporated by reference.

### BACKGROUND OF THE INVENTION

1. Field of the Invention

The present invention relates to nasal pharmaceutical products containing peptide active agents in formulations that include nasal bioavailability enhancers. In particular, citrates, fatty acids, sugar esters of fatty acids or acyl carnitines are used in certain nasal peptide formulations, especially in preferred combinations discussed herein.

2. Description of the Related Art

Given their size and molecular structure, peptides are frequently administered by subcutaneous or intramuscular injection. Other routes of administration can be technically difficult because peptides tend to be poorly absorbed through tissue and readily degraded by bodily fluids. Oral administration, for example, can be problematic due to degradation of the peptide active agent by stomach or intestinal proteases.

Nasal delivery is also frequently plagued by low bioavailabilty of the active agent. Even where nasal delivery is possible, manufacturing costs can be undesirably high because of the large concentration of active agent required to provide clinical efficacy in view of low bioavailability occasioned by the difficulty of peptides crossing the nasal mucosa.

In U.S. Pat. No. 6,440,392, a nasal salmon calcitonin formulation is disclosed having certain concentrations of citric acid and/or citric acid salt. In U.S. Pat. No. 5,759,565, a nasal calcitonin formulation containing benzalkonium chloride is disclosed. There remains, however, a need for further improving the bioavailability of nasally administered peptides in a formulation that is well-tolerated by the nasal mucosa.

### SUMMARY OF THE INVENTION

It is accordingly an object of the present invention to provide peptide pharmaceutical compositions which, when administered nasally, provide good bioavailability, resulting in a significant increase in blood concentration of the peptide.

It is another object of the invention to provide peptide pharmaceutical compositions that are well-tolerated when administered to the nasal mucosa.

In one embodiment, the invention provides a pharmaceutical composition for nasal delivery of a peptide active agent comprising:
(1) said active agent; and
(2) a bioavailability enhancing agent selected from the group consisting of a fatty acid, a sugar ester of a fatty acid and a mixture thereof.

In another embodiment, the invention provides a pharmaceutical composition for nasal delivery of a peptide active agent comprising:
(1) said active agent;
(2) a sugar ester of a fatty acid; and
(3) an acyl carnitine.

In another embodiment, the invention provides a pharmaceutical composition for nasal delivery of a peptide active agent comprising:
(1) said active agent;
(2) oleic acid;
(3) sucrose laurate;
(4) a citrate-based bioavailability enhancing agent selected from the group consisting of citric acid, citric acid salt and a mixture of citric acid and citric acid salt;

wherein said pharmaceutical composition is an aqueous solution buffered at a pH no lower that 3.0 and no higher than 6.5.

In another embodiment, the invention provides a pharmaceutical composition for nasal delivery of a peptide active agent comprising:
(1) said active agent;
(2) L-lauroyl carnitine;
(3) sucrose laurate;
(4) a citrate-based bioavailability enhancing agent selected from the group consisting of citric acid, citric acid salt and a mixture of citric acid and citric acid salt;

wherein said pharmaceutical composition is an aqueous solution buffered at a pH no lower than 3.0 and no higher than 6.5.

The invention is further explained by the following non-limiting description of preferred embodiments.

### DETAILED DESCRIPTION OF THE INVENTION

### Peptide Active Ingredients

Peptide active ingredients which may benefit from nasal delivery in accordance with the invention include any therapeutic agent that is physiologically active and has, as part of its molecular structure, a plurality of amino acids and at least one peptide bond. In addition to natural amino acids, the amino acids may be D-amino acids or unnatural amino acids, some examples of which are discussed infra. The molecular structure may further include other substituents or modifications. For example, salmon calcitonin is amidated at its C-terminus, as is a preferred parathyroid hormone truncate that is the subject of experimental data infra. Some peptides may be amidated at locations that are not amidated in nature, or may be otherwise modified.

Peptide active compounds of the invention include, but are not limited to, insulin, vasopressin, calcitonin (including not only salmon calcitonin, but other calcitonins as well). Other examples include calcitonin gene-related peptide, parathyroid hormone (including amidated or unamidated truncates thereof such as PTH1-31-amide or PTH1-34-amide), desmopressin, luteinizing hormone-releasing factor, erythropoietin, tissue plasminogen activators, human growth hormone, adrenocorticototropin, various interleukins, enkephalin, and the like. Many others are known in the art.

Both man-made and natural peptides can be delivered nasally in accordance with the invention. Thus, the peptide active compound, in some embodiments, could be glucagon-like peptide-1 (GLP-1), or analogs thereof, desmopressin (DDAVP), leuprolide, 2,6-dimethyltyrosine-D-arginine-phenylalanine-lysine amide (DMT-DALDA), peptidomimetics and the like.

The peptides for use in the invention may be in free form or in pharmaceutically acceptable salt or complex form, e.g., in pharmaceutically acceptable acid addition salt form. Such salts and complexes are known and tend to possess an equivalent degree of activity and tolerability to the free forms. Suitable acid addition salt forms for use in accordance with the invention include for example the hydrochlorides and acetates.

### Enhancement of Bioavailability

Enhancement of bioavailability is achieved with one or more classes of enhancers selected from fatty acids, sugar esters of fatty acids, acyl carnitines and citrates. It is preferred to use combinations thereof, except that acyl carnitines and fatty acids are not used together because of undesirable interaction between them. Preferred molecular structures regarding each class is discussed below.

### Fatty Acids

Without intending to be bound by theory, it is believed that the fatty acids interact with peptides to desirably enhance their ability to penetrate cell membranes, thus enhancing transcellular transport. The hydrophobic region of fatty acids is believed important to this function, and should desirably include as many consecutive carbon atoms as possible, consistent with water solubility, preferably at least 8 consecutive carbon atoms, especially 10-14 carbon atoms. Preferred fatty acids include but are not limited to lauric acid and oleic acid. When used, preferred concentration of fatty acid is between 0.1 and 4.0 mg/mL, especially between 0.5 and 2.0 mg/mL.

### Sugar Esters of Fatty Acids

Without intending to be bound by theory, it is believed that the sugar esters of fatty acids may interact with cells in a manner that could alter their shape, increase pore size, and thereby desirably increase paracellular transport. They may also provide benefit in transcellular transport. When fatty acids and sugar esters of fatty acids are used in combination, bioavailability may be especially enhanced by the combination of enhanced transcellular and enhanced paracellular transport. Like the fatty acids, the hydrophobic region should also preferably include at least 8 consecutive carbon atoms, especially 10-14 carbon atoms. The sugar moiety may aid water solubility. Preferred sugar esters of fatty acids include but are not limited to sucrose laurate, glucose laurate and fructose laurate. When used, preferred concentration of sugar esters of fatty acids is between 0.1 and 10.0 mg/mL, especially between 0.5 and 5.0 mg/mL.

### Acyl Carnitines

Acyl carnitines are believed to enhance bioavailability, and in preferred embodiments are combined with a sugar ester of a fatty acid. Preferred acyl carnitines include but are not limited to L-lauroyl carnitine and myristoyl carnitine. When used, preferred concentration of acyl carnitine is between 0.1 and 10.0 mg/mL, especially between 0.5 and 5.0 mg/mL.

### Citrates

Citrate-type bioavailability enhancing agents selected from the group consisting of citric acid, citric acid salt and mixtures thereof are preferably used in combination with one or more of the other enhancers discussed herein. Without intending to be bound by theory, it is believed that citrate-type enhancing agents may increase paracellular transport. Preferably, the concentration of all such citrate-type enhancing agents, when used in the invention, will be no lower than 5 mM and no higher than 50 mM, more preferably 10-25 mM. Without intending to be bound by theory, it is believed that shelf stability may be undesirably reduced at higher citrate concentrations due to interaction of citrate with the active peptide at the amino terminus of the peptide, or at lysyl side chains.

### Other Preferences

The above defined compositions may be applied in accordance with the invention to the nasal mucosa, e.g. either in drop or in spray form. As hereinafter described however, they are most preferably applied in spray form, i.e., in the form of finely divided droplets.

The compositions of the invention may of course also include additional ingredients, in particular components belonging to the class of conventional pharmaceutically applicable surfactants.

Preferably, the liquid pharmaceutical composition of the present invention contains a pharmaceutically acceptable diluent or carrier suitable for application to the nasal mucosa. Aqueous saline may be used for example.

The compositions of the invention are formulated so as to permit administration via the nasal route. For this purpose they may also contain, e.g. minimum amounts of any additional ingredients or excipients desired, for example, additional preservatives or, e.g. ciliary stimulants such as caffeine.

Generally for nasal administration a mildly acid pH will be preferred. Preferably the compositions of the invention have a pH of from about 3.0 to 6.5.

The compositions of the invention should also possess an appropriate isotonicity and viscosity. Preferably they have an osmotic pressure of from about 260 to about 380 mOsm/liter. Desired viscosity for the nasal spray is preferably less than 0.98 cP.

Compositions in accordance with the present invention may also comprise a conventional surfactant, preferably a non-ionic surfactant. When a surfactant is employed, the amount present in the compositions of the invention will vary depending on the particular surfactant chosen, the particular mode of administration (e.g. drop or spray) and the effect desired. In general, however, the amount present will be of the order of from about 0.1 mg/ml to about 10 mg/ml, preferably about 0.5 mg/ml to 5 mg/ml and most preferably about 1 mg/ml.

Preferably, a pharmaceutically acceptable preservative is included. Many are known in the art, and have been used in the past in connection with aqueous nasal pharmaceuticals. For example, benzyl alcohol or phenyethyl alcohol or a mixture thereof may be employed. In one embodiment, 0.2% phenylethyl alcohol and 0.5% benzyl alcohol are used in combination.

The amount of peptide to be administered, and hence the amount of active ingredient in the composition of the invention will, of course, depend on the particular peptide chosen, the condition to be treated, the desired frequency of administration and the effect desired.

The quantity of the total composition administered at each nasal application suitably comprises from about 0.05 to 0.15 ml, typically about 0.1 ml.

For the purposes of nasal administration, the compositions of the invention will preferably be kept in a container provided with means enabling application of the contained composition to the nasal mucosa, e.g. put up in a nasal applicator device. Suitable applicators are known in the art and include those adapted for administration of liquid compositions to the nasal mucosa in drop or spray form. Because dosing should be as accurately controlled as possible, use of spray applicators for which the administered quantity is susceptible to precise regulation will generally be preferred. Suitable administrators include, e.g. atomizing devices, pump-atomizers and aerosol dispensers. In the latter case, the applicator will contain a composition in accordance with the invention together with a propellant medium suitable for use in a nasal applicator. The atomizing device will be provided with an appropriate spray adaptor allowing delivery of the contained composition to the nasal mucosa. Such devices are well known in the art.

The container, e.g., nasal applicator, may contain sufficient composition for a single nasal dosing or for the supply of several sequential dosages, e.g. over a period of days or weeks. Quantities of individual dosages supplied will preferably be as hereinbefore defined.

Set forth below are some non-limiting examples of several formulations in accordance with the invention, together with efficacy data.

### Experimental Design:

Female Sprague-Dawley rats, weighing between 225 and 250 g, were used in these studies. Rats were fasted overnight prior to administration of the test substance, but were allowed free access to water. Rats were anesthetized with a combination of ketamine and xylazine and a canula was inserted into the carotid artery for blood sampling. The volume of each blood sample collected was 0.5 mL.

A 20 μL dose was administered by touching the left nostril with the disposable tip of an Eppendorf micropipette and gently applying pressure to the plunger of the pipette. Blood samples were collected prior to dosing and at 10, 20, 40, 60 and 120 minutes after the administration of PTH(1-34)NH₂, (1-2 mg/mL) in 0.85% sodium chloride containing a formulation as indicated in the tables. PTH(1-34)NH₂ is a parathyroid hormone truncate whose molecular structure includes only the first 34 amino acids of natural human parathyroid hormone wherein the C-terminal amino acid is amidated.

The concentration of PTH(1-34)NH₂ in plasma was determined using an ELISA. Briefly, the assay consists of incubating rat samples in 96 well ELISA plates that were coated with rabbit antibody to PTH(1-34)NH₂. After incubating and washing the plates, goat antibody to PTH(1-34)NH₂ was added to the plates. Bound antibody was detected with rabbit anti-goat IgG-horse-raddish conjugate and 3,3′,5,5′-Tetramethylbenzidine peroxide substrate after washing off unbound goat antibody. The amount of PTH(1-34)NH₂ in blood samples was directly proportional to the yellow color in the wells.

### Results:

### Table 1: Effect of Enhancer on Intranasal Absorption of PTH(1-34)NH₂

Rats were given intranasal PTH(1-34)NH₂(1-2 mg/mL) in 16 mM sodium phosphate/8 mm citric acid (pH 4.8) containing 0.85% sodium chloride and the indicated final concentration of enhancer.

| | Mean | |
|---|---|---|
| | Cmax | |
| Enhancer | pg/mL** | sem |
| 0.1% Tween 80 (17)* | 57,244 | 11,037 |
| 0.2% LLC (17) | 203,341 | 35,544 |
| 0.5% LLC (12) | 218,637 | 19,457 |
| 0.2% SL (14) | 118,455 | 13,304 |
| 0.4% SL (3) | 101,127 | 27,553 |
| 0.5% SL (6) | 135,551 | 18,331 |
| 1.0% SL (11) | 225,742 | 40,825 |

*(n) number of rats
**Corrected to 1 mg dose.

In the above table, “LLC” means L-lauroyl carnitine and “SL” means sucrose laurate. The results in Table 1 show that the replacement of 0.1% Tween 80 with 0.2% LLC increased the mean Cmax of PTH(1-34)NH₂ at least 3 fold, and that increasing the amount of LLC to 0.5% did not further increase the mean Cmax of PTH(1-34)NH₂. Replacing 0.1% Tween 80 with 0.2% SL increased the Cmax of PTH(1-34)NH₂ 2 fold. Adding up to 0.5% SL did not further increase the mean Cmax; however, when 1% SL was included in the formulation, the mean Cmax increased nearly 4 fold.

Table 2: Effect of Mixing Oleic Acid with Sucrose Laurate on Intranasal Absorption of PTH(1-34)NH₂.

Rats were given intranasal PTH(1-34)NH₂ (1 mg/mL) in 20 mm citric acid/sodium citrate (pH 3.8) containing 0.85% sodium chloride and the indicated final concentration of enhancer. Sodium oleate was added to the formulation prior to the addition of citrate buffer.

| | Mean | |
|---|---|---|
| | Cmax | |
| Enhancer | pg/mL** | sem |
| 0.1% Tween 80 (3)* | 38,221 | 6,536 |
| 0.1% Tween 80 + 0.1% SL (3) | 74,495 | 12,849 |
| 0.1% Tween 80 + 0.1% SL + 0.1% oleic acid (3) | 99,599 | 31,452 |

**(n) number of rats
**Corrected to 1 mg dose.

In the above table, “SL” means sucrose laurate. The results summarized in Table 2 show that the addition of sucrose laurate to the formulation increased the Cmax of PTH(1-34)NH₂ nearly 2 fold and the inclusion sodium oleate increased the Cmax of PTH(1-34)NH₂.2.6 fold. At pH 3.8 sodium oleate exists as oleic acid, which is insoluble in water. To overcome this problem, oleic acid was added to the formulation as sodium oleate prior to the addition of citrate buffer.

Although the present invention has been described in relation to particular embodiments thereof, many other variations and modifications and other uses will become apparent to those skilled in the art. Therefore, the present invention is limited not by the specific disclosure herein, but only by the claims herein.

What is claimed is:

1. A pharmaceutical composition for nasal delivery of a peptide active agent comprising:
(1) said active agent; and
(2) a bioavailability enhancing agent selected from the group consisting of a fatty acid, a sugar ester of a fatty acid and a mixture thereof.

2. The pharmaceutical composition of claim 1, wherein both a fatty acid and a sugar ester of a fatty acid are present.

3. The pharmaceutical composition of claim 1, wherein said fatty acid has a hydrophobic region of at least eight consecutive carbon atoms in its molecular structure.

4. The pharmaceutical composition of claim 1, wherein said fatty acid has a hydrophobic region of from ten to fourteen consecutive carbon items in its molecular structure.

5. The pharmaceutical composition of claim 1, wherein said pharmaceutical composition is an aqueous solution buffered at a pH no lower than 3.0 and no higher than 6.5.

6. The pharmaceutical composition of claim 1, wherein said composition further includes a citrate-type bioavailability enhancing agent selected from the group consisting of citric acid, citric acid salt and a mixture thereof.

7. The pharmaceutical composition of claim 6, wherein the total combined concentration of all citrate-based bioavailability enhancing agent is no lower than 5 mM and no higher than 50 mM.

8. The pharmaceutical composition of claim 2, wherein said composition further includes a citrate-type bioavailability enhancing agent selected from the group consisting of citric acid, citric acid salt and a mixture thereof.

9. The pharmaceutical composition of claim 8, wherein the total combined concentration of all citrate-based bioavailability enhancing agent is no lower than 5 mM and no higher than 50 mM.

10. The pharmaceutical composition of claim 1, wherein said peptide includes an amide group as part of its molecular structure.

11. The pharmaceutical composition of claim 1, wherein said fatty acid is selected from a group consisting of lauric acid and oleic acid.

12. The pharmaceutical composition of claim 1, wherein said sugar ester is selected from a group consisting of sucrose laurate, glucose laurate and fructose laurate.

13. The pharmaceutical composition of claim 2, wherein said fatty acid is oleic acid and said sugar ester is sucrose laurate.

14. A pharmaceutical composition for nasal delivery of a peptide active agent comprising:
(1) said active agent;
(2) a sugar ester of a fatty acid; and
(3) an acyl carnitine.

15. The pharmaceutical composition of claim 14, wherein said sugar ester is sucrose laurate.

16. The pharmaceutical composition of claim 14, wherein said sugar ester has a hydrophobic region of at least eight consecutive carbon atoms in its molecular structure.

17. The pharmaceutical composition of claim 14, wherein said sugar ester has a hydrophobic region of from ten to fourteen consecutive carbon items in its molecular structure.

18. The pharmaceutical composition of claim 14, wherein said pharmaceutical composition is an aqueous solution buffered at a pH no lower than 3.0 and no higher than 6.5.

19. The pharmaceutical composition of claim 14, wherein said composition further includes a citrate-type bioavailability enhancing agent selected from the group consisting of citric acid, citric acid salt and a mixture thereof.

20. The pharmaceutical composition of claim 20, wherein the total combined concentration of all citrate-based bioavailability enhancing agents is no lower than 5 mM and no higher than 50 mM.

21. The pharmaceutical composition of claim 16, wherein said composition further includes a citrate-type bioavailability enhancing agent selected from the group consisting of citric acid, citric acid salt and a mixture thereof.

22. The pharmaceutical composition of claim 21, wherein the total combined concentration of all citrate-based bioavailability enhancing agent is no lower than 5 mM and no higher than 50 mM.

23. The pharmaceutical composition of claim 14, wherein said peptide includes an amide group as part of its molecular structure.

24. The pharmaceutical composition of claim 14, wherein said acyl carnitine is selected from a group consisting of L-lauroyl carnitine and myristoyl carnitine.

25. The pharmaceutical composition of claim 14, wherein said sugar ester is selected from a group consisting of sucrose laurate, glucose laurate and fructose laurate.

26. The pharmaceutical composition of claim 14, wherein said acyl carnitine is L-lauroyl carnitine and wherein said sugar ester is sucrose laurate.

27. A pharmaceutical composition for nasal delivery of a peptide active agent comprising:
(1) said active agent;
(2) oleic acid;
(3) sucrose laurate;
(4) a citrate-based bioavailability enhancing agent selected from the group consisting of citric acid, citric acid salt and a mixture of citric acid and citric acid salt;
wherein said pharmaceutical composition is an aqueous solution buffered at a pH no lower than 3.0 and no higher than 6.5.

28. A pharmaceutical composition for nasal delivery of a peptide active agent comprising:
(1) said active agent;
(2) L-lauroyl carnitine;
(3) sucrose laurate;
(4) a citrate-based bioavailability enhancing agent selected from the group consisting of citric acid, citric acid salt and a mixture of citric acid and citric acid salt;
wherein said pharmaceutical composition is an aqueous solution buffered at a pH no lower than 3.0 and no higher than 6.5.

29. The pharmaceutical composition of claim 1 wherein the peptide active agent is PTH(1-34)NH₂.

30. The pharmaceutical composition of claim 14 wherein the peptide active agent is PTH(1-34)NH₂.

31. The pharmaceutical composition of claim 27 wherein the peptide active agent is PTH(1-34)NH₂.

32. The pharmaceutical composition of claim 28 wherein the peptide active agent is PTH(1-34)NH₂.
